(19) United States
(12) Patent Application Publication
(10) Pub. No.: US 2003/0072819 A1
(43) Pub. Date: Apr. 17, 2003

(54) Compositions and methods for enhancing drug delivery

(76) Inventor: Yuanjin Tao, Fremont, CA (US)

Correspondence Address: Karen R. Zachow, Morrison & Foerster LLP, 755 Page Mill Road, Palo Alto, CA, 94304-1018, US

(21) Appl. No.: 10/141,528

(22) Filed: May 6, 2002

Related U.S. Application Data
(60) Provisional application No. 60/288,615, filed on May 4, 2001.

(51) Int. Cl. A61K 35/78; A61K 33/04; A61K 33/32; A61K 31/375; A61K 31/355; A61K 31/198

(52) U.S. Cl. 424/725; 424/728; 424/764; 424/757; 424/769; 514/458; 514/474; 514/565; 424/702; 424/642

(57) ABSTRACT

Compositions and methods for enhancing the delivery of another composition, enhancing blood circulation and/or enhancing cellular metabolism are provided. The compositions comprise herbals, natural nutritional supplements, minerals and/or vitamins.

### CROSS-REFERENCE TO RELATED APPLICATIONS

This application claims the priority benefit of U.S. Provisional application No. 60/288,615, filed May 4, 2001, which is hereby incorporated by reference in its entirety.

### TECHNICAL FIELD

This invention relates to compositions and methods for enhancing delivery of a composition of interest to a target site in an individual. More specifically, it relates to nutraceutical compositions and methods for enhancing the delivery of a composition of interest.

### BACKGROUND ART

One of the biggest challenges in drug development is presented by inefficiencies in delivering a drug to its target site. Efficiency of delivery can affect the dosage that needs to be administered in order for the drug to be of an efficacious concentration/amount when it reaches its target site. A drug that is administered locally may not cross a tissue barrier efficiently, or may undergo an undesirable amount of degradation due to delay in arriving at its target site. A drug that is administered systemically may suffer an unacceptable degree of inactivation, degradation and/or dilution as it makes it way to its target site. One school of thought proposes that improving systemic circulation, microcirculation, and/or cellular metabolism could improve the efficiency of the drug delivery process.

Various herbal substances have been studied for their potential in improving blood circulation or cellular metabolism. Jiang & Qian, Chung Kuo Yao Li Hsueh Pao (1995), 16(5):399-402; Wu & Sun, Chung Kuo Yao Li Hsueh Pao (1992), 13(6):520-3; Zhao et al., Chung Kuo Chung HsiI Chieh Ho Tsa Chih (1994), 14(2):71-3, 67; Wang & Ma, Chung Hsi I Chiech Ho Tsa Chih (1990), 10(2):101-2, 70; Jia & Tang, Chung Hsi I Chieh Ho Tsa Chih (1991), 11(4):199-202, 195; Goto et al., Planta Med. (1996), 62(5):436-9; Xue et al., Chung Kuo Chung Yao Tsa Chih (1993), 18(10):621-3, 640; Yoshikawa et al., Chem. Pharm. Bull (Tokyo) (1997), 45(6):1039-45; Liu et al., J. Ethnopharmacol. (1994), 42(3):183-91; Qi, ChungHsi I Chieh Ho Tsa Chih (1991), 11(2):102-4, 69; Fu, ChungKuo Chung Hsi I Chieh Ho Tsa Chih (1992), 12(4):228-9, 198; Zee-Cheng, Methods Find. Exp. Clin. Pharmacol. (1992), 14(9):725-36; Zee-Cheng, Methods Find. Exp. Clinc. Pharmacol. (1992), 14(9):725-36; Wang & Han, Yao Hsueh Hsueh Pao (1993), 28(8):572-6; Haraguchi et al., Bioorg. Med. Chem. (1998), 6(3):339-47; Haraguchi et al., J. Pharm. Pharmacol. (2000), 52(2):219-23; Ju et al., Yao Hsueh Hsueh Pao (1989), 24(1 1):807-12; Hu et al., Biomed. Environ. Sci. (1999), 12(1):10-4; Luper, Altern. Med. Rev. (1999), 4(3):178-88; Shim et al., Planta Med. (2000), 66(1):40-3; Acharya et al., Indian J Med. Res. (1993), 98(-HD-):69-74; Yokozawa et al., Phytomedicine (2000), 6(6):439-45; Mokhova et al., FEBSLett. (1991), 289(2):187-9; Visentin et al J Pharmacol. Exp. Ther. (1995), 275(2):1069-75; Newall et al., Herbal Medicine: A Guide for Health Care Professionals (1996), (London, Pharmaceutical Press); Duke, Handbook of Medicinal Herbs (1985), (Boca Raton, CRC Press); Ratnakumari et al., Metabolism (1993), 42(8):1039-46; Berthon P et al., Comp. Biochem. Physiol. A Physiol. (1997), 117(1):141-5; Jacob & Belleville, Pathol. Biol. (Paris) (1992), 40(9):910-9; Kobayashi et al., Jpn. Circ. J (1992), 56(1):86-94; Arduini et al., Transfusion (1997), 166-74; Postiglione et al., Int. J Clin. Pharmacol. Res. (1990), 10(1-2):129-32; Postiglione et al., Pharmacol. Res. (1991), 23(3):241-6. The substances studied were found to provide varying degrees of effects on blood circulation and cellular metabolism.

It would clearly be advantageous to have herbal and natural nutritional supplements that can enhance the delivery of another composition, preferably a therapeutic composition. The invention described and claimed in this specification provides this advantage by presenting compositions and methods for enhancing the delivery of another composition, enhancing blood circulation and enhancing cellular metabolism.

All references cited herein, including patent applications and publications, are incorporated by reference in their entirety.

### DISCLOSURE OF THE INVENTION

Compositions and methods for enhancing the delivery of another composition (preferably a therapeutic composition) to a target site in an individual are provided. Compositions and methods for enhancing blood circulation or cellular metabolism are also provided. The compositions comprise substances in quantities that are effective for enhancing the delivery of compositions that are administered in conjunction with the compositions of the invention. Without wishing to be bound by theory, the compositions of the invention enhance delivery of another composition at least in part by increasing blood circulation (in particular, microcirculation) and/or cellular metabolism. In one aspect, methods of enhancing delivery of a composition comprising administration of the claimed compositions are provided. In another aspect, methods of enhancing blood circulation, preferably microcirculation, comprising administration of the claimed compositions are provided. In yet another aspect, methods of enhancing cellular metabolism comprising administration of the claimed compositions are provided.

Accordingly, in one aspect, the invention provides a first composition for enhancing the delivery of a second composition, said first composition comprising two substances selected from the group consisting of a member of the panax (such as noto ginseng) or eleutherococus genus, a member of the paeonia genus (such as peonia lactiflora Pall. (Bai Shao)), L-camitine, a member of the glycyrrhiza genus (such as licorice root) and black cohosh.

In another aspect, the invention provides a first composition for enhancing the delivery of a second composition, said first composition comprising three substances selected from the group consisting of a member of the panax (such as noto ginseng) or eleutherococus genus, a member of the paeonia genus (such as peonia lactiflora Pall. (Bai Shao)), L-camitine, a member of the glycyrrhiza genus (such as licorice root) and black cohosh.

In another aspect, the invention provides a first composition for enhancing the delivery of a second composition, said first composition comprising four substances selected from the group consisting of a member of the panax (such as noto ginseng) or eleutherococus genus, a member of the paeonia genus (such as peonia lactiflora Pall. (Bai Shao)), L-camitine, a member of the glycyrrhiza genus (such as licorice root) and black cohosh.

In another aspect, the invention provides a first composition for enhancing the delivery of a second composition, said first composition comprising a member of the panax (such as noto ginseng) or eleutherococus genus, a member of the paeonia genus (such as peonia lactiflora Pall. (Bai Shao)), L-carnitine, a member of the glycyrrhiza genus (such as licorice root) and black cohosh.

In still another aspect, the invention provides a composition selected from the group consisting of the first compositions of the aspects of the invention described in the preceding paragraphs, further comprising at least one (preferably 1, 2, 3, 4, 5, 6 or 7) substances selected from the group consisting of a member of the Daemonorops genus (such as xuejie), a member of the corydalis genus (such as yanhusuo), white willow bark, vitamin E, vitamin C and a mineral (such as zinc and selenium).

In yet another aspect, the invention provides a method of making a composition of the invention, said method comprising combining two or more (for example, 2, 3, 4 or 5) substances (preferably in an effective amount) selected from the group consisting of a member of the panax (such as noto ginseng) or eleutherococus genus, a member of the paeonia genus (such as peonia lactiflora Pall. (Bai Shao)), L-camitine, a member of the glycyrrhiza genus (such as licorice root) and black cohosh. In some embodiments, the method further comprises combining said two or more substances with at least one (for example, 1, 2, 3, 4, 5, 6 or 7) substance (preferably in an effective amount) selected from the group consisting of a member of the Daemonorops genus (such as xuejie), a member of the corydalis genus (such as yanhusuo), white willow bark, vitamin E, vitamin C and a mineral (such as zinc and selenium). In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

In another aspect, the invention provides a method for enhancing the delivery of a second composition comprising administering a composition selected from the aspects of the invention described in the preceding paragraphs to an individual to whom the second composition is also administered, whereby delivery of the second composition is enhanced. In some embodiments of this aspect of the invention, the first composition further comprises at least one (for example, 1, 2, 3, 4, 5, 6 or 7) substance selected from the group consisting of a member of the Daemonorops genus (such as xuejie), a member of the corydalis genus (such as yanhusuo), white willow bark, vitamin E, vitamin C and a mineral (such as zinc and selenium).

In yet another aspect, the invention provides a method for enhancing blood circulation (preferably microcirculation) comprising administering a composition selected from the aspects of the invention described in the preceding paragraphs. In some embodiments of this aspect of the invention, the first composition further comprises at least one (for example, 1, 2, 3, 4, 5, 6 or 7) substance selected from the group consisting of a member of the Daemonorops genus (such as xuejie), a member of the corydalis genus (such as yanhusuo), white willow bark, vitamin E, vitamin C and a mineral (such as zinc and selenium). In still another aspect, the invention provides a method for enhancing cellular metabolism comprising administering a composition selected from the aspects of the invention described in the preceding paragraphs. In some embodiments of this aspect of the invention, the first composition further comprises at least one (for example, 1, 2, 3, 4, 5, 6 or 7) substance selected from the group consisting of a member of the Daemonorops genus (such as xuejie), a member of the corydalis genus (such as yanhusuo), white willow bark, vitamin E, vitamin C and a mineral (such as zinc and selenium).

### MODES FOR CARRYING OUT THE INVENTION

The present invention discloses compositions comprising two or more substances that in combination are effective for enhancing the delivery of another composition. The invention also provides methods for enhancing delivery of a composition of interest comprising administering a composition of this invention in conjunction with the composition of interest. Methods of enhancing blood circulation (preferably microcirculation) and cellular metabolism are also provided.

### Definitions

The term “delivery” of a composition (such as a therapeutic composition), and variations thereof, as used in this specification, generally refers to transfer of a composition of interest from the site of introduction (administration) of the composition to the target site(s) in the individual. A target site is any location in the body where the composition of interest can manifest an intended effect (which includes, for example, a therapeutic effect, an enhancement effect, or a prophylactic effect). A target site can be a general location in the body of the individual (for example, the central nervous system, the lymphatic system), or a more specific location, such as, for example, an organ, a tissue, a gland, a cell, a sub-cellular compartment, or a molecule in the body of the individual. There may be one or more target sites in an individual with respect to a delivered composition.

The term “delivered composition,” “composition to be delivered,” “composition that is delivered,” “second composition”, “composition of interest” and variations thereof, as used in this specification, refers to a composition the delivery of which to a target site(s) in an individual is to be enhanced by the administration of the compositions of the present invention. These terms, as used herein, can refer to a single compound or substance, or a mixture of 2 or more compounds or substances. In a preferred embodiment, a delivered composition is a therapeutic composition. A “therapeutic composition” is a composition administered to achieve a treatment or therapeutic effect. A therapeutic composition can be, for example, a composition comprising a pharmaceutical compound such as a drug.

The term “treating,” “treatment,” and variations thereof, as used in this specification, refers to an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a therapeutic composition.

A “treatment effect” or “therapeutic effect” is manifested if there is a change in the condition being treated, as measured by the criteria constituting the definition of the terms “treating” and “treatment.” There is a “change” in the condition being treated if there is at least 10% improvement, preferably at least 25%, more preferably at least 50%, even more preferably at least 75%, and most preferably at least 100%. The change can be based on improvements in the severity of the treated condition in an individual, or on a difference in the frequency of improved conditions in populations of individuals with and without treatment with the therapeutic compositions with which the compositions of the present invention are administered in combination.

The term “enhancing” delivery of a composition (such as a therapeutic composition), and variations thereof, as used in this specification, refers to an increase in the delivery of a composition above that normally obtained when the composition is administered without the compositions of the invention. “An increase in the delivery of a composition” is manifested when there is an acceleration of rate of arrival of a composition at a target site and/or increase in amount of composition that localizes to a target site. It can also manifest as an extension of the duration that a composition remains at a target site. It can also manifest wherein less of a composition of interest has to be administered to an individual when it is administered in conjunction with a composition of the invention in order to achieve a similar effect as when a composition of interest is not administered in conjunction with a composition of the invention. The enhancing effect preferably, but not necessarily, results in treatment of symptoms for which a particular dosage of the delivered composition of interest alone is not effective or is less effective therapeutically. Enhancement of delivery is achieved when there is at least a 10% increase in the delivery of a composition. Preferably the increase is at least 25%, more preferably at least 50%, even more preferably at least 75%, most preferably at least 100%. Enhancement of delivery can also be deemed to have been achieved when there is at least a 10% increase in the effect (generally and preferably therapeutic effect) of a composition of interest when it is administered in conjunction with a composition of the present invention compared to when it is administered alone. Preferably the increase is at least 25%, more preferably at least 50%, even more preferably at least 75%, most preferably at least 100%.

“An increase in the therapeutic effects” is manifested when there is an acceleration and/or increase in intensity and/or extent of the therapeutic effects obtained with a therapeutic composition. It also includes extension of the longevity of therapeutic benefits. It can also manifest where a lower amount of the therapeutic composition is required to obtain the same benefits and/or effects when it is co-administered with compositions provided by the present invention as when a higher amount of the therapeutic composition is administered alone. The enhancing effect preferably, but not necessarily, results in treatment of acute symptoms for which the therapeutic composition alone is not effective or is less effective therapeutically.

“Blood circulation,” as used herein, generally refers to the course of the blood from the heart through the arteries, capillaries, and veins back again to the heart. “Microcirculation,” as used herein, refers to the circulation of blood in and/or through microvessels (such as capillaries) and/or circulation of blood within a tissue/organ. Blood circulation or microcirculation is “enhanced” when at least one of the criteria that define blood circulation and/or microcirculation is enhanced. These criteria are known in the art, for example, plasma viscosity, blood pressure and blood viscosity. There is enhancement of any of these criteria when there is preferably at least 10%, more preferably at least 30%, even more preferably at least 50%, and most preferably at least 75% increase in the measurement of any of these criteria.

“Metabolism,” “cellular metabolism,” and “intracellular metabolism,” as used herein, refers to metabolic reactions and/or processes in the body, as understood in the art. It generally refers to the sum of the chemical and/or physical changes occurring in tissue, consisting of anabolism (those reactions that convert small molecules into large) and catabolism (those reactions that convert large molecules into small), including both endogenous large molecules as well as biodegradation of xenobiotics. Metabolism, cellular metabolism, and intracellular metabolism is “enhanced” when at least one of the criteria that define metabolism is enhanced. These criteria are known in the art, for example, production rate/level of pyruvate, acetyl CoA, and adenosine triphosphate. There is enhancement of any of these criteria when there is preferably at least 10%, more preferably at least 30%, even more preferably at least 50%, and most preferably at least 75% increase in the measurement of any of these criteria.

An “effective amount” is an amount of a composition or substance(s) sufficient to enhance delivery of a composition that is to be delivered after one or more administrations of that amount. An effective amount can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

“Co-administering” or “co-administration” of compositions, and “administered in conjunction with”, as used herein, refers to the administration of a composition of the present invention and a composition of interest within a certain time period. The time period is preferably 12 hours, more preferably 6 hours, still more preferably 3 hours. These terms most preferably mean the compositions are administered together.

“Individual,” as used herein, refers to a vertebrate, preferably a mammal, more preferably a human.

A “genus,” as used herein, refers to the botanical classification term as understood in the art. As used herein, it can also include botanical entities that are closely related to a genus (but are not officially classified under said genus) with respect to their relevant function(s) in the compositions of the invention.

“Xuejie,” as used herein, refers to extracts of xuejie. It is also known as sanguis draconis and Daemonorops droco B1. A member of the genus Daemonorops is preferably selected from the group consisting of Daemonorops draco B 1 (xuejie), Dracaena cambodiana Pierre, Dracaena cinnabari Balf, Dracaena draco Linn., Dracaena schizantha Baker, Dracaena dihynophyllus, Dracaena micracanthus Becc., Dracaena propinqus Becc., Dracaena draconcellus Becc., Dracaena motleyi Becc., Dracaena sparsiflorus Becc., Pterocarpus draco Linn., Croton draco Schlecht, Croton hibiscifolius kunth, Croton sanguifluus H. B. et K Nov. and Croton gossypiifolius Vahl.

“Yanhusuo,” as used herein, refers to extracts of yanhusuo. It is also known as Rhizoma corydalis yanhusuo and Corydalis yanhusuo W. T. Wang. A member of the genus Corydalis is preferably selected from the group consisting of Corydalis yanhusuo W. T. Wang (yanhusuo), Corydalis amabilis Migo, Corydalis amabigua Cham. et Schlecht. Var amurensts Maxim, Corydalis turtschaninovii Bess., Corydalis schangini (Pall.) B., Corydalis glaucescens Rgl. and Corydalis ledebouriana Kar. Et Kir.

“Baishaoyao,” as used herein, refers to extracts of baishaoyao. It is also known as Radix paeoniae lactiflorae and paeonia lactiflora Pall. A member of the genus Paeonia is preferably selected from the group consisting of Paeonia lactiflora Pall. (Bai shao) and paeonia veitchii Lynch.

“Shanqi,” as used herein, refers to extracts of shanqi. It is also known as Radix Notoginsheng and Panax notoginsheng (Burk.) F. H. Chen. A member of the genus Panax is preferably selected from the group consisting of Panax notoginseng (Burk.) F.H. Chen (Shanqi), Panax pseudo-ginseng Wall.var.notoginseng (Burk) Hoo et Tseng, Panax ginseng C.A.Mey (P.schinseng Nees) and Panax guinquefolium L. A member of the genus eleutherococus is preferably Acanthopanax sentocosus (Rupr. et Maxim.) or Eleutherococus senticosus (Rupr. et Maxim.).

“Gancao,” as used herein, refers to extracts of gancao. It is also known as Radix glycyrrhizae uralensis, Glycyrrhiza uralensis Fischer or licorice root. A member of the glycyrrhiza genus is preferably selected from the group consisting of Glycyrrhiza uralensis Fisch (Gan cao), Glycyrrhiza glabra L., Glycyrrhizainflata Batal., Glycyrrihiza korshiskyi G. Hrig., Glycyrrhiza aspera Pall and Glycyrrhiza yunnanensis Cheng f. et L. K. Tai.

“White willow bark,” as used herein, refers to extracts of white willow bark. It is also known as Saliz alba caerulea.

“Black cohosh root,” as used herein, refers to extracts of black cohosh root. It is also known as rhizome of Cimicifuga racemosa or Cimicifuga racemosa.

“L-camitine,” as used herein, refers to the biochemical molecule known as such by persons of skill in the art. It is generally known as a zwitterionic compound formed from lysine. It also refers to related molecules such as L-acetyl-carnitine.

“Vitamin C,” as used herein, refers to ascorbic acid and salts thereof.

“Vitamin E,” as used herein, refers to D alpha-tocopherol, preferably in succinate form.

“Zinc,” as used herein, refers to the form(s) of the mineral known to persons of skill in the art to be therapeutically effective in the body of the individual. It is preferably provided as zinc gluconate.

“Selenium,” as used herein, refers to the form(s) of the mineral known to persons of skill in the art to be therapeutically effective in the body of the individual. It is preferably provided as selenium aminoate.

“Extract,” as used herein, refers to the substances obtained from the specified source plant, or parts thereof (for e.g., root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed under conditions of high pressure, preferably from 0.5 to 12 bar, more preferably 1 to 10 bar, most preferably 3 to 7 bar, and preferably at elevated temperatures (preferably within a range of 15° C. to 120° C., more preferably 30° C. to 100° C., most preferably 45° C. to 75° C.). The extract is preferably treated to yield a form suitable for mixing of two or more substances. The form is preferably a dried powder. The powder form is yielded from preferably at least about a 1: 10, more preferably at least about a 1:8, most preferably at least about a 1:5 concentrate of the starting solution. Concentration to powder form is preferably achieved by evaporation to yield a dried powder form. The extracts used in this invention can also be obtained from commercial sources such as Sun Ten Laboratories (Irvine, Calif.), Qualiherb (Cerritos, Calif.), Mayway (Oakland, Calif.), Ming Tong Herb (Oakland, Calif.) and Acta (Sunnyvale, Calif.). It is understood that any method or conditions known in the art to yield extracts comparable in effectiveness in enhancing delivery of another composition, enhancing blood circulation and/or enhancing cellular metabolism to those produced by the preceding preferred extraction method can be used for the purposes of this invention.

### Formulation of the Composition

Each substance contained in the compositions provided by this invention is provided in an amount that lies within specific quantitative ranges herein disclosed to be effective for enhancing the delivery of a second composition, enhancing blood circulation (preferably microcirculation) and/or enhancing cellular metabolism.

According to the present invention, an effective amount of a composition comprises preferably 50 mg to 3000 mg, more preferably 500 mg to 2000 mg, most preferably 800 mg to 1500 mg of a member of the Panax genus (such as shanqi) or Eleutherococus genus (such as Acanthopanax senticosus or Eleutherococus senticosus); preferably from 50 mg to 1000 mg, more preferably 200 mg to 800 mg, most preferably 400 mg to 600 mg of a member of the Paeonia genus (such as baishaoyao); preferably 30 mg to 600 mg, more preferably 120 mg to 500 mg, most preferably 250 mg to 400 mg L-camitine; preferably 50 mg to 1000 mg, more preferably 200 mg to 800 mg, most preferably 400 mg to 600 mg of a member of the Glycyrrhiza genus (such as gancao); and/or preferably 5 mg to 150 mg, more preferably 40 mg to 120 mg, most preferably 75 mg to 100 mg black cohosh root.

In the various embodiments of the invention, the compositions further comprise preferably from 100 to 1500 mg, more preferably 300 mg to 1200 mg, most preferably 600 mg to 900 mg of a member of the Daemonorops genus (such as xuejie); and/or preferably 50 mg to 1000 mg, more preferably 200 mg to 800 mg, most preferably 400 mg to 600 mg of a member of the Corydalis genus (such as yanhusuo); preferably 5 mg to 150 mg, more preferably 10 mg to 120 mg, most preferably 75 mg to 100 mg white willow bark; preferably 10 mg to 250 mg, more preferably 80 mg to 300 mg, most preferably 120 mg to 200 mg vitamin E; preferably 20 mg to 400 mg, more preferably 80 mg to 300 mg, most preferably 120 mg to 200 mg vitamin C; preferably 10 mg to 80 mg, more preferably 20 mg to 60 mg, most preferably 30 mg to 50 mg zinc; and preferably 5 mg to 60 mg, more preferably 10 mg to 50 mg, most preferably 20 mg to 40 mg selenium.

According to this invention, the compositions can be formulated in whatever form that retains the efficacy of the compositions for enhancing the delivery of another composition, enhancing blood circulation and/or enhancing cellular metabolism. Preferably, the compositions are packaged in the form of capsules. The capsules are preferably of size “0”, “00”, “000”, “1”, “2”, “3” or “4.” A preferred method for packaging into capsules involves mixing substances (extracts, vitamin and minerals) that are preferably in powder form. The substances are preferably mixed to at least 30%, more preferably to at least 60%, even more preferably to at least 90% mixture consistency, and most preferably to homogeneity. The substances in powder form are provided in the initial mixture at ratios according to the effective quantities disclosed above. Methods for mixing the substances are known in the art, including, but not limited to, stirring, agitation or vibration achieved manually or through the aid of a machine. A preferred mixing machine is a V-mixer, preferably of 100 to 1400-liter size, more preferably of 150 to 1300-liter size, and most preferably of 200 to 1200-liter size. Preferably, the resulting powder mixture is filtered to screen out particulates (i.e., anything that a person of skill in the art would recognize to be larger than powder size). A preferred filter is a 1/20-inch particle size filter. Preferably, the filtered mixture is packaged into capsules according to the weight desired for each capsule. Preferably, the capsule is of size “00”. The weight of mixture per capsule is preferably from 5 mg to 1000 mg, more preferably 100 mg to 800 mg, even more preferably 400 mg to 700 mg. It is understood that other physical forms of the compositions of this invention suitable for administration to an individual can also be used, including, for example, tablets, salves or liquids, as long as the compositions can be delivered to the target tissues in the body where the compositions in the preferred form described above exert their effects.

The ingredients of the compositions can be mixed with pharmaceutically acceptable solvents, excipients and/or filler substances. These materials are known in the art, and are described in sources such as Remington's Pharmaceutical Sciences, 18th Edition, Mack Publishing (1990).

### Administration of Compositions of the Present Invention

Compositions in any of the forms described above can be administered by any method known to one of skill in the art, but oral administration is preferred. The compositions are preferably administered in capsule form.

An effective amount of a composition is provided preferably in from 1 to 8 administrations, more preferably in from 2 to 6 administrations, and most preferably in from 3 to 5 administrations. Administration of an effective amount is preferably completed within 24 hours. A composition can be ingested alone, or with any other substance, such as a liquid, that aids ingestion of the compositions. Ingestion of the compositions can be before or after food consumption. The compositions of the present invention can be combined with any second composition, preferably a therapeutic composition, to enhance the delivery of the second composition.

A second composition is any composition with respect to which enhancement of delivery to its target site(s) in the body is desired. For example, a second composition can be a drug the efficacy of which can be increased by enhancing the rate of its delivery to its target site(s). In another example, a second composition can be a drug for which decreasing its toxicity would be desirable. In this example, enhancement of delivery to the drug's target site would be advantageous by decreasing the initial amount of the drug that has to be administered, due to more efficient delivery to the target site.

In some embodiments, the present compositions are administered in conjunction with a second composition that is effective in treating eye discomfort syndromes, pains and/or discomfort in joints such as the wrist, pains and/or discomfort in the neck and/or shoulder, or pains and/or discomfort in the back and leg. Compositions of the present invention are particularly effective in enhancing the delivery of the compositions disclosed in co-pending patent applications entitled “Compositions And Methods For Treating Eye Discomfort” (WO 01/91765, WO 01/91769, WO 01/91770), “Compositions And Methods For Treating Hand And Wrist Discomfort” (WO01/91766), “Compositions and Methods For Treating Neck And Shoulder Discomfort” (WO 01/91778) and “Compositions and Methods For Treating Back And Leg Discomfort” (WO 01/91771), which are hereby incorporated by reference. The present compositions enhance the delivery, and hence the therapeutic effects of these compositions for treating conditions such as eye discomfort syndromes, wrist strain or pain, neck or shoulder pain, and back or leg pain.

Other medical conditions for which the compositions of the present invention are particularly useful include post-trauma muscle spasms, bleeding (internal and external), sciatic pain, swelling and bleeding due to injury and bruising, eye conditions/diseases such as cataract and macular degeneration, general pain (treatable with pain medication), high/low blood pressure, cardiovascular disease, arthritis, memory loss/sub-optimal memory, allergy Type I-IV, sinusitis, attention deficit disorder, menstrual pain, menopause, enlarged prostate and sexual dysfunction. The present compositions enhance the delivery of compositions used to treat these conditions.

A second composition may or may not be provided in the same form as the compositions of the invention. Thus, for example, the composition of the invention may be provided in capsule form, and a second composition is provided in aerosol form. A composition of the invention and a second composition may be administered by the same route or different routes of administration. Thus, for example, one may be administered orally while another is administered by injection, or one is administered by spraying onto a mucosal surface while another is introduced orally.

The timing of administration of the composition of the invention and a second composition can be varied according to the needs of the individual, or in accordance with the empirical determination or experience of the health care professional or individual. In some embodiments, the composition of the invention is administered prior to administration of the second composition. In these embodiments, the composition of the invention is administered preferably at least 7 days, more preferably at least 5 days, even more preferably at least 3 days, and most preferably at least 0.5 day prior to administration of the second composition. In some embodiments, the composition of the invention is administered preferably at least 12 hours, more preferably at least 9 hours, even more preferably at least 6 hours, and most preferably at least 3 hours prior to administration of the second composition. In other embodiments, the composition of the invention is administered after the administration of the second composition. In these embodiments, the composition of the invention is administered preferably at least 7 days, more preferably at least 5 days, even more preferably at least 3 days, and most preferably at least 0.5 day after administration of the second composition. In some of these embodiments, the composition of the invention is administered preferably at least 12 hours, more preferably at least 9 hours, even more preferably at least 6 hours, and most preferably at least 3 hours after administration of the second composition. In some embodiments, the composition of the invention and the second composition are administered at the same time. In still other embodiments, the composition of the invention is administered before, with and after the administration of the second composition.

Enhancement of delivery of a second composition can be assessed by any methods known in the art, and as described herein. For example, the amount of a second composition that is delivered to a target site when the second composition is administered with or without a composition of the invention can be determined biochemically using tests appropriate for the target site, as known in the art. In another example, enhancement of delivery can be assessed based on enhancement of the effects of the second composition. Thus, in this example, enhancement of delivery of a therapeutic composition would be determined based on enhancement of therapeutic effects.

Enhancement of blood circulation can be assessed by any of a variety of methods known in the art. For example, blood circulation can be measured by impedance cardiogram, pheopneumogram, hemorheologic examination (such as for blood viscosity, plasma viscosity, hematocrit, blood pressure and erythrocyte electrophoresis), and blood gaseous analysis. See, for e.g., Jia & Tang, Chung His I Chiech Ho Tsa Chih (1991), 11(4):199-202, 195.

Enhancement of cellular metabolism can be assessed by any of a variety of methods known in the art. For example, cellular metabolism can be measured by measuring the levels of metabolism-related products such as ammonia, glutamine, glutamate, pyruvate, lactate, alpha-ketoglutarate, adenosine triphosphate, free coenzyme A and acetyl CoA, and adenosine triphosphate production rate in mitochondria. See, for e.g., Ratnakumari et al., Metabolism (1993), 42(8):1039-46; and Berthon et al., Comp. Biochem. Physiol. A. Physiol. (1997), 117(1):141-5.

The following Examples are provided to illustrate, but not limit, the invention.

### EXAMPLES

### Example 1

### An Illustrative Example of the Formulation of A Single “00” Capsule, and the Production Thereof

A composition capable of enhancing the delivery of a second composition contains substances in the indicated quantities as listed in Table 1.

TABLE 1

| SUBSTANCE [Commercial Source] | AMOUNT (mg) |
|---|---|
| Xuejie [Min Tong Herb, Oakland, CA] | 133 |
| Yanhusuo [Mayway, Oakland, CA] | 83 |
| Baishaoyao [Qualiherb, Cerritos, CA] | 83 |
| Shanqi [Sun Ten, Irvine, CA] | 250 |
| Gancao [Qualiherb, Cerritos, CA] | 83 |
| White willow bark [Acta, Sunnyvale, CA] | 10 |
| Black cohosh root [Acta, Sunnyvale, CA] | 10 |
| L-carnitine [Acta, Sunnyvale, CA] | 50 |
| Vitamin E (D Alpha-Tocopherol | 21.19 |
| succinate) [Acta, Sunnyvale, CA] | |
| Vitamin C [Acta, Sunnyvale, CA] | 30 |

Capsules containing the composition above are manufactured according to the method used by a commercial manufacturer, such as Acta (Sunnyvale, Calif.). Briefly, the substances listed above, in powder form and obtained from the commercial sources such as those indicated in Table 1, are mixed in input amounts in accordance to the ratio of the substances in the composition as a whole. Mixing is accomplished with a V-mixer, grinding for 15 to 30 minutes, at a speed of 15 to 30 rpm (rounds per minute), to produce a homogenous mixture of the input substances. Particulates (non-powder forms) are then filtered out with a 1/20-inch particle size filter that separates particulates from the powder. 671 mg of the filtered mixture is then packaged into each size “00” capsule.

### EXAMPLE 2

Illustrative Example of Enhancement Of Delivery of A Composition For Treating Eye Discomfort

A randomized, double-blinded and controlled study is performed. Individuals suffering from eye discomfort such as dry eyes are divided into three groups. Individuals in the first group are administered the composition set forth in Table 2. The composition of Table 2 is exemplified as Example 1 of co-pending patent application entitled “Compositions And Methods For Treating Eye Discomfort” (WO 01/91765, WO 01/91769, WO 01/91770; which are hereby incorporated herein by reference in their entirety). Individuals in the second group are administered the composition exemplified in Example 1 of the instant specification. Individuals in the third group are co-administered the compositions administered to the individuals of the first and second group. Preferably, a fourth group is included in which individuals are administered a placebo that does not contain any of the substances found in the compositions administered to the first and second group.

TABLE 2
A Composition For Treating Eye Discomfort

| SUBSTANCE [Commercial source] | AMOUNT (mg) |
|---|---|
| Fuling [Qualiherb, Cerritos, CA] | 16.7 |
| Jueminzi [Qualiherb, Cerritos, CA] | 8.3 |
| Sang ye [Qualiherb, Cerritos, CA] | 33.3 |
| Shanyao [Qualiherb, Cerritos, CA] | 16.7 |
| Shanzhuyu [Mayway, Oakland, CA] | 16.7 |
| Shudihaung [Qualiherb, Cerritos, CA] | 100.0 |
| Bilberry [Acta, Sunnyvale, CA] | 16.7 |
| Danshen [Qualiherb, Cerritos, CA] | 16.7 |
| Gouqizhi [Qualiherb, Cerritos, CA] | 100.0 |
| Juhua [Qualiherb, Cerritos, CA] | 66.7 |
| Baijili [Qualiherb, Cerritos, CA] | 16.7 |
| Beta-carotene [Acta, Sunnyvale, CA] | 22.5 |
| Copper gluconate [Acta, Sunnyvale, CA] | 7.1 |
| Gujincao [Qualiherb, Cerritos, CA] | 8.3 |
| Magnesium stearate NF [Acta, Sunnyvale, CA] | 10.0 |
| Mudanpi [Qualiherb, Cerritos, CA] | 16.7 |
| Rice flour powder [Acta, Sunnyvale, CA] | 8.3 |
| Seleniuum aminoate [Acta, Sunnyvale, CA] | 6.7 |
| Vitamin B-2 (riboflavin) [Acta, Sunnyvale, CA] | 5.0 |
| Vitamin C (ascorbic acid) [Acta, Sunnyvale, CA] | 66.7 |
| Vitamin E (D Alpha-tocopherol | 76.0 |
| succinate) [Acta, Sunnyvale, CA] | |
| Zexie [Acta, Sunnyvale, CA] | 16.7 |
| Zinc gluconate [Acta, Sunnyvale, CA] | 57.7 |

Individuals in the first group are daily administered from 1 to 8 capsules of the composition of Table 2. Individuals in the second group are daily administered from 1 to 8 of the capsules of Example 1 of the instant specification. Individuals in the third group are daily administered the combined dosages of the individuals of the first and second group.

At least 20 individuals are tested, randomly assigned in approximately equal number to the various groups. Individuals are evaluated to suffer from a particular eye discomfort symptom, such as dry eyes.

The study is carried out for at least 1 week. During the treatment course, a dosage amount selected from the range of 1 to 8 capsules of each composition is administered to each individual once or multiple times daily, not exceeding 6 capsules of each composition per day. Capsules are administered before or after food consumption.

A clinical questionnaire is used to evaluate individuals' eye discomfort symptoms. A clinical coordinator and/or physician evaluates the individuals' eye discomfort symptoms and fills out the questionnaire. Evaluation can include ophthalmologic tests such as the Vision test, the Tear Breakup test, the Zone Quick test, the Rose Bengal test and/or cornea staining and injection. Evaluation can also include self-evaluation by individuals. Evaluations can be performed daily, or more or less frequently depending on statistical or clinical (ability to detect or track symptomatic improvements) need.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales); and (4) non-efficacy, as defined as no improvement in symptoms.

The results of the groups are compared to assess any enhanced therapeutic effects due to the co-administration of the composition exemplified in Example 1. Enhancement of therapeutic effects can be indicative of enhancement of delivery of the therapeutic composition.

### Example 3

### Illustrative Example of Enhancement Of Delivery of A Composition For Treating Wrist Discomfort

A randomized, double-blinded and controlled study is performed. Individuals suffering from wrist discomfort such as wrist pain are divided into three groups. Individuals in the first group are administered the composition set forth in Table 3. The composition of Table 3 is exemplified as Example 1 of co-pending patent application entitled “Compositions And Methods For Treating Hand And Wrist Discomfort” (WO 01/91766; which is hereby incorporated herein by reference in its entirety). Individuals in the second group are administered the composition exemplified in Example 1 of the instant specification. Individuals in the third group are co-administered the compositions administered to the individuals of the first and second group. Preferably, a fourth group is included in which individuals are administered a placebo that does not contain any of the substances found in the compositions administered to the first and second group.

TABLE 3
A Composition For Treating Hand And Wrist Discomfort

| SUBSTANCE [commercial source] | AMOUNT (mg) |
|---|---|
| Baishaoyao [Qualiherb, Cerritos, CA] | 33.3 |
| Fangfeng [Qualiherb, Cerritos, CA] | 33.3 |
| Guizi [Qualiherb, Cerritos, CA] | 50 |
| Honghua [Qualiherb, Cerritos, CA] | 33.3 |
| Chuanxiong [Qualiherb, Cerritos, CA] | 50 |
| Yuanhusuo [ Mayway, Oakland, CA] | 50 |
| Dangquiwei [Mayway, Oakland, CA] | 50 |
| Gancao [Qualiherb, Cerritos, CA] | 33.3 |
| Ginger [Qualiherb, Cerritos, CA] | 33.3 |
| Huangqi [Mayway, Oakland, CA] | 133 |
| Sangzhi [Qualiherb, Cerritos, CA] | 66.7 |
| Yinyanghuo [Qualiherb, Cerritos, CA] | 33.3 |
| Weilingxian [Mayway, Oakland, CA] | 66.7 |
| Dazao [Mayway, Oakland, CA] | 16.7 |
| Vitamin B-1 (Thiamine) [Acta, Sunnyvale, CA] | 4.2 |
| Vitamin B-2 (Riboflavin) [Acta, Sunnyvale, CA] | 4.2 |
| Vitamin B-3 (Niacin) [Acta, Sunnyvale, CA] | 8.3 |
| Vitamin B-5 (Pantothenic acid) [Acta, | 4.2 |
| Sunnyvale, CA] | |
| Vitamin B-6 (pyridoxine) [Acta, | 33 |
| Sunnyvale, CA] | |
| Vitamin C (ascorbic acid) [Acta, | 100 |
| Sunnyvale, CA] | |
| Vitamin E (D alpha-tocopherol | 14.1 |
| succinate) [Acta, Sunnyvale, CA] | |
| Beta-carotene [Acta, Sunnyvale, CA] | 7.5 |
| Zinc gluconate [Acta, Sunnyvale, CA] | 21 |
| Quercetin [Acta, Sunnyvale, CA] | 8.3 |
| Selenium aminoate [Acta, Sunnyvale, CA] | 6.7 |
| Rice flour powder [Acta, Sunnyvale, CA] | 10 |
| Magnesium stearate NT [Acta, Sunnyvale, CA] | 10 |

Individuals in the first group are daily administered from 1 to 8 capsules of the composition of Table 3. Individuals in the second group are daily administered from 1 to 8 of the capsules of Example 1 of the instant specification. Individuals in the third group are daily administered the combined dosages of the individuals of the first and second group.

At least 20 individuals are tested, randomly assigned in approximately equal number to the various groups. Individuals are evaluated to suffer from a particular wrist discomfort symptom, such as wrist pain.

The study is carried out for at least 1 week. During the treatment course, a dosage amount selected from the range of 1 to 8 capsules of each composition is administered to each individual once or multiple times daily, not exceeding 6 capsules of each composition per day. Capsules are administered before or after food consumption.

A clinical questionnaire is used to evaluate individuals' wrist discomfort symptoms. A clinical coordinator and/or physician evaluates the individuals' wrist discomfort symptoms and fills out the questionnaire. Evaluations can be performed daily, or more or less frequently depending on statistical or clinical (ability to detect or track symptomatic improvements) need.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales); and (4) non-efficacy, as defined as no improvement in symptoms.

The results of the groups are compared to assess any enhanced therapeutic effects due to the co-administration of the composition exemplified in Example 1. Enhancement of therapeutic effects can be indicative of enhancement of delivery of the therapeutic composition.

### Example 4

### Illustrative Example of Enhancement Of Delivery of A Composition For Treating Back and Leg Discomfort

A randomized, double-blinded and controlled study is performed. Individuals suffering from back or leg discomfort such as back or leg pain are divided into three groups. Individuals in the first group are administered the composition set forth in Table 4. The composition of Table 4 is exemplified as Example 1 of co-pending patent application entitled “Compositions And Methods For Treating Back And Leg Discomfort” (WO 01/91771; which is hereby incorporated herein by reference in its entirety). Individuals in the second group are administered the composition exemplified in Example 1 of the instant specification. Individuals in the third group are co-administered the compositions administered to the individuals of the first and second group. Preferably, a fourth group is included in which individuals are administered a placebo that does not contain any of the substances found in the compositions administered to the first and second group.

TABLE 4
A Composition For Treating Back And Leg Discomfort

| SUBSTANCE [commercial source] | AMOUNT (mg) |
|---|---|
| Duzhong [Mayway, Oakland, CA] | 33 |
| Huainiuxi [Qualiherb, Cerritos, CA] | 33 |
| Jixueteng [Qualiherb, Cerritos, CA] | 117 |
| Qiannianjian [Qualiherb, Cerritos, CA] | 33 |
| Rougui [Qualiherb, Cerritos, CA] | 50 |
| Xiquancao [Qualiherb, Cerritos, CA] | 133 |
| Jinyingzi [Mayway, Oakland, CA] | 33 |
| Tougucao [Qualiherb, Cerritos, CA] | 33 |
| Duhuo [Qualilierb, Cerritos, CA] | 100 |
| Weilingxian [Mayway, Oakland, CA] | 67 |
| Shenjincao [Qualiherb, Cerritos, CA] | 17 |
| Vitamin B-1 [Acta, Sunnyvale, CA] | 5 |
| Vitamin B-2 [Acta, Sunnyvale, CA] | 5 |
| Vitamin B-6 [Acta, Sunnyvale, CA] | 15 |
| Vitamin B-12 [Acta, Sunnyvale, CA] | 0.0125 |
| Vitamin C (ascorbic acid) [Acta, Sunnyvale, CA] | 30 |
| Vitamin E (D Alpha-Tocopherol) [Acta, | 21.19 |
| Sunnyvale, CA] | |
| Devil's Claw [Acta, Sunnyvale, CA] | 10 |
| Black cohosh [Acta, Sunnyvale, CA] | 10 |
| Selenium [Acta, Sunnyvale, CA] | 20 |

Individuals in the first group are daily administered from 1 to 8 capsules of the composition of Table 4. Individuals in the second group are daily administered from 1 to 8 of the capsules of Example 1 of the instant specification. Individuals in the third group are daily administered the combined dosages of the individuals of the first and second group.

At least 20 individuals are tested, randomly assigned in approximately equal number to the various groups. Individuals are evaluated to suffer from a particular back or leg discomfort symptom, such as back or leg pain.

The study is carried out for at least 1 week. During the treatment course, a dosage amount selected from the range of 1 to 8 capsules of each composition is administered to each individual once or multiple times daily, not exceeding 6 capsules of each composition per day. Capsules are administered before or after food consumption.

A clinical questionnaire is used to evaluate individuals' back or leg discomfort symptoms. A clinical coordinator and/or physician evaluates the individuals' back or leg discomfort symptoms and fills out the questionnaire. Evaluations can be performed daily, or more or less frequently depending on statistical or clinical (ability to detect or track symptomatic improvements) need.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales); and (4) non-efficacy, as defined as no improvement in symptoms.

The results of the groups are compared to assess any enhanced therapeutic effects due to the co-administration of the composition exemplified in Example 1. Enhancement of therapeutic effects can be indicative of enhancement of delivery of the therapeutic composition.

### Example 5

### Illustrative Example of Enhancement Of Delivery of A Composition For Treating Neck And Shoulder Discomfort

A randomized, double-blinded and controlled study is performed. Individuals suffering from neck or shoulder discomfort such as neck or shoulder pain are divided into three groups. Individuals in the first group are administered the composition of Table 5. The composition of Table 5 is exemplified as Example 1 of co-pending patent application entitled “Compositions and Methods For Treating Neck And Shoulder Discomfort” (WO 01/91778; which is hereby incorporated herein by reference in tis entirety). Individuals in the second group are administered the composition exemplified in Example 1 of the instant specification. Individuals in the third group are co-administered the compositions administered to the individuals of the first and second group. Preferably, a fourth group is included in which individuals are administered a placebo that does not contain any of the substances found in the compositions administered to the first and second group.

TABLE 5
A Composition For Treating Neck And Shoulder Discomfort

| | |
|---|---|
| SUBSTANCE [commercial source] | AMOUNT (mg) |

| | |
|---|---|
| Huangqi [Mayway, Oakland, CA] | 33 |
| Dangquiwei [Mayway, Oakland, CA] | 33 |
| Weilingxian [Qualiherb, Cerritos, CA] | 67 |
| Gegen [Qualiherb, Cerritos, CA] | 167 |
| Guizi [Qualiherb, Cerritos, CA] | 167 |
| Baishaoyao [Qualiherb, Cerritos, CA] | 167 |
| Gancao [Qualiherb, Cerritos, CA] | 17 |
| Dazao [Mayway, Oakland, CA] | 17 |
| Bilberry [Acta, Sunnyvale, CA] | 10 |
| Ginger [Qualiherb, Cerritos, CA] | 17 |
| Vitamin B-1 (thiamine) [Acta, Sunnyvale, CA] | 5 |
| Vitamin B-2 (riboflavin) [Acta, Sunnyvale, CA] | 5 |
| Vitamin B-6 (pyridoxine) [Acta, Sunnyvale, CA] | 15 |
| Vitamin B-12 (cobalamin) [Acta, Sunnyvale, CA] | 0.0125 |
| Vitamin C (ascorbic acid) [Acta, Sunnyvale, CA] | 30 |
| Vitamin E (D alpha-tocopherol | 21.19 |
| succinate) [Acta, Sunnyvale, CA] | |
| White willow bark [Acta, Sunnyvale, CA] | 10 |
| Quercitin [Acta, Sunnyvale, CA] | 25 |
| Selenium aminoate [Acta, Sunnyvale, CA] | 20 |

Individuals in the first group are daily administered from 1 to 8 capsules of the composition of Table 5. Individuals in the second group are daily administered from 1 to 8 of the capsules of Example 1 of the instant specification. Individuals in the third group are daily administered the combined dosages of the individuals of the first and second group.

At least 20 individuals are tested, randomly assigned in approximately equal number to the various groups. Individuals are evaluated to suffer from a particular neck or shoulder discomfort symptom, such as neck or shoulder pain.

The study is carried out for at least 1 week. During the treatment course, a dosage amount selected from the range of 1 to 8 capsules of each composition is administered to each individual once or multiple times daily, not exceeding 6 capsules of each composition per day. Capsules are administered before or after food consumption.

A clinical questionnaire is used to evaluate individuals' neck or shoulder discomfort symptoms. A clinical coordinator and/or physician evaluates the individuals' neck or shoulder discomfort symptoms and fills out the questionnaire. Evaluations can be performed daily, or more or less frequently depending on statistical or clinical (ability to detect or track symptomatic improvements) need.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales); and (4) non-efficacy, as defined as no improvement in symptoms.

The results of the groups are compared to assess any enhanced therapeutic effects due to the co-administration of the composition exemplified in Example 1. Enhancement of therapeutic effects can be indicative of enhancement of delivery of the therapeutic composition.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

What is claimed is:

1. A first composition for enhancing the delivery of a second composition, said first composition comprising two substances selected from the group consisting of noto ginseng, peonia, L-camitine, licorice root and black cohosh.

2. A first composition for enhancing the delivery of a second composition, said first composition comprising three substances selected from the group consisting of noto ginseng, peonia, L-camitine, licorice root and black cohosh.

3. A first composition for enhancing the delivery of a second composition, said first composition comprising four substances selected from the group consisting of noto ginseng, peonia, L-carnitine, licorice root and black cohosh.

4. A first composition for enhancing the delivery of a second composition, said first composition comprising noto ginseng, peonia, L-carnitine, licorice root and black cohosh.

5. A method for enhancing the delivery of a second composition comprising administering to an individual to whom said second composition is administered an effective a mount of a first composition selected from the group consisting of the first compositions of claims 1 to 4, whereby delivery of said second composition is enhanced.

6. The method of claim 5, wherein said first composition further comprises at least one substance selected from the group consisting of xuejie, yanhusuo, white willow bark, vitamin E, vitamin C, zinc and selenium.

7. A method for enhancing blood circulation comprising administering to an individual an effective amount of a first composition selected from the group consisting of the first compositions of claims 1 to 4, whereby blood circulation is enhanced.

8. The method of claim 7, wherein said first composition further comprises at least one substance selected from the group consisting of xuejie, yanhusuo, white willow bark, vitamin E, vitamin C, zinc and selenium.

9. A method for enhancing cellular metabolism comprising administering to an individual an effective amount of a first composition selected from the group consisting of the first compositions of claims 1 to 4, whereby cellular metabolism is enhanced.

10. The method of claim 9, wherein said first composition further comprises at least one substance selected from the group consisting of xuejie, yanhusuo, white willow bark, vitamin E, vitamin C, zinc and selenium.

11. A method of making a composition for enhancing the delivery of a second composition, said method comprising combining at least two substances selected from the group consisting of noto ginseng, peonia, L-camitine, licorice root and black cohosh.

12. The method of claim 11, said method further comprising combining said at least two substances with at least one substance selected from the group consisting of xuejie, yanhusuo, white willow bark, vitamin E, vitamin C, zinc and selenium.
